(19) United States
(12) Patent Application Publication
(10) Pub. No.: US 2005/0064013 A1
(43) Pub. Date: Mar. 24, 2005

(54) Ready to drink beverage composition having liquid form HGH pre-cursor and method of making same

(76) Inventor: Jeffrey Liebrech, Columbus, OH (US)

Correspondence Address: Jeffrey C. Maynard, 154 Barbara Road, Severna Park, MD, 21146, US

(21) Appl. No.: 10/351,221

(22) Filed: Jan. 24, 2003

Related U.S. Application Data
(60) Provisional application No. 60/417,765, filed on Oct. 11, 2002.

(51) Int. Cl. A61K 47/00; A61K 33/24; A61K 33/34; A61K 31/198

(52) U.S. Cl. 424/439; 514/561; 514/565; 424/638; 424/654; 424/656

(57) ABSTRACT

This invention relates to novel compositions and methods for creating such composition that can be ingested by humans as a dietary supplement that can be used by athletes and bodybuilders for restoring human growth hormones. The novel compositions comprise fluids containing water, sugar including the carbohydrates, fructose and maltodextrin, and electrolytes chromium, copper, potassium, magnesium, sodium, and citric acid. The composition also includes selected amino acids such as leucine, isoleucine, valine and arginine.

### CROSS REFERENCE TO RELATED APPLICATION

This Application claims the benefit of U.S. Provisional Application, Ser. No. 60/417,765 entitled Beverage Compositions for Use in Rehydration and Nutrition During Athletic Exercise and Methods of Making Same, filed with the U.S. Patent and Trademark Office on Oct. 11, 2002.

### BACKGROUND OF THE INVENTION

1. Field of the Invention

This invention relates to a nutritional dietary supplement drink containing a liquid form human growth hormone pre-cursor, in particular, the invention relates to a drink for consumption on a daily basis by athletes of all skill levels to aid muscle growth.

2. Background if the Prior Art

There are presently a substantial number of electrolyte drinks on the market that are alleged to replenish essential electrolytes and water lost from the body during physical activity. Physical exercise can be distinguished in different categories i.e. those requiring strength, strength and speed or endurance. In practice, this can be heavy work, muscle activity under severe conditions (e.g. high temperature, high altitude), leisure sports or athletic performance.

Athletes who participate in sports at any level, amateur or professional, strive to bring their bodies to a physical state that is optimum for the sport or activity of interest. One factor that enables athletes to participate effectively is a high degree of development of the aerobic capacity and/or strength of skeletal muscle. The inherent problem is a built-in failure in stimulus of production and release of human growth hormone (HGH). The natural production of HGH incrementally decreases in the normal human being after approximately 23 years of age.

The desire to attain, in a rapid manner, the maximum degree of skeletal muscle adaptation to exercise has led some athletes to resort to the use of drugs. Such drugs, particularly steroids, are known to “force” muscle growth to degrees greater than can be achieved by exercise and diet alone. The use of such drugs is both illegal and dangerous. The side effects of steroids are dangerous and unacceptable. Indeed, all professional sports leagues, as well as collegiate athletics, have a long list of potentially performance enhancing substances, many of which may be over-the-counter products, the use of any of which is banned. It is therefore important to provide a natural growth stimulus that does not contain any of the banned ingredients.

Muscle requires a large array of nutrients, including amino acids (which are derived from protein) for growth. Such nutrients have been supplied by ingesting diets that provide the necessary amounts of protein (the source of amino acids), calories and other nutrients.

Powders that contain the appropriate amino acids are available in cans and jars that come with scoopers for measuring dosages into a glass to which water or other beverage is to be added. The problems with this prior art are: consuming the powder dry is very difficult, and many of the powders taste bad, even if mixed with water; if mixed with juice or milk, the fructose or lactose of the juice/milk raises blood glucose and insulin levels. Such products are, however, generally unpalatable and do not, in fact, satisfy the body completely by replenishing all the essential constituents which are lost.

One object of the present invention, then, is to provide a new method of stimulating production of HGH for natural muscle growth stimulation, in the form of a tasty, convenient ready-to-drink beverage. The provision of an agonist/inhibition blockade to HGH Releasing Factor is the premise of the use of L-Arginine at basal metabolic levels. Until now, these were only available in a powder barely palatable form because of technical and biochemical difficulty in creating a product that is bioavailable. To stimulate HGH production in this manner goes against the teaching of the prior art because it was believed not to be possible to combine the necessary amino acids with any food for the reasons stated above. The delivery system of the present invention therefore combines the preferred amino acid dosages with ready-to-drink beverages that taste good and are convenient, as opposed to the prior art of powders and numerous pills.

The present invention provides a multi-electrolyte composition including water, fructose, inulin, and flavoring to provide a more palatable drink with a better balance of electrolytes and HGH. This isotonic energy drink also includes critical vitamins and minerals as important nutrients for muscle growth and optimum performance.

### SUMMARY OF THE INVENTION

The invention comprises a ready to drink beverage product for direct increase of human growth hormone (HGH). Simple sugars and critical vitamins and minerals are blended in water to provide an electrolyte solution for daily consumption to stimulate muscle growth.

It is, therefore, an object of the present invention to enable beverage compositions for use in growth stimulation that avoids the disadvantages of the prior art.

It is another object of the present invention to enable a method for preparing such beverage compositions.

It is yet another object of the present invention to enable a beverage composition that stimulates production of HGH that typically diminishes with age.

It is more specifically an object of the invention to provide such a beverage composition that is pleasant tasting.

It is another object of the invention to provide a beverage composition having electrolytes in correct physiological quantities to increase the efficiency, of the body to utilize sugars and improve muscular activities.

It is still a further object of the present invention to enable a beverage composition that does not contain ingredients that have been banned from use by professional athletes.

In accordance with the above and other objects, a nutritional dietary supplement drink containing a liquid form human growth hormone pre-cursor, containing optimum quantities of sugars, slats and minerals, including essential amino acids is disclosed. In particular, a drink for consumption on a daily basis by athletes of all skill levels to aid muscle growth is described.

The various features of novelty that characterize the invention will be pointed out with particularity in the claims of this application.

### DETAILED DESCRIPTION OF THE INVENTION

The invention summarized above may be better understood by referring to the following description. This description of an embodiment, set out below to enable one to build and use an implementation of the invention, is not intended to limit the enumerated claims, but to serve as a particular example thereof. Those skilled in the art should appreciate that they may readily use the conception and specific embodiments disclosed as a basis for modifying or designing other methods and systems for carrying out the same purposes of the present invention. Those skilled in the art should also realize that such equivalent assemblies do not depart from the spirit and scope of the invention in its broadest form.

In a first embodiment, a ready to drink beverage having a liquid form HGH pre-cursor is mixed together using water, sweeteners, citric acid, flavoring and a blend of vitamins and minerals. The combination of vitamins and minerals includes Pantothenic Acid, Boron, Chromium, Copper, and Choline. Additional ingredients include L-Arginine, L-Isoleucine, L-Leucine, and L-Valine.

In a preferred embodiment, a powder premixture is formulated comprising the following composition:

| | | |
|---|---|---|
| Pantothenic Acid (as D-Calcium Pantothenate, USP) | 2-8 | mg |
| Boron (as Sodium Borate, USP) | 0.5-2 | mg |
| Chromium (as Chromium Chloride (6H₂O), USP) | 10-15 | mcg |
| Copper (as Copper Sulfate) | 0.1-0.4 | mg |
| L-Isoleucine (USP) | 15-45 | mg |
| L-Leucine (USP) | 30-75 | mg |
| L-Valine (USP) | 15-45 | mg |
| Choline (as Choline Bitartrate, FCC) | 15-45 | mg |
| Maltodextrin (as Maltrin M-100) | Q.S. | |

Approximately 225 mg of the premixture is used for an 8 fluid ounce serving. Preferably, the premixture contains:

| | | |
|---|---|---|
| Pantothenic Acid (as D-Calcium Pantothenate, | approximately 5 | mg |
| USP) | | |
| Boron (as Sodium Borate, USP) | approximately 1 | mg |
| Chromium (as Chromium Chloride (6H₂O), | approximately 12 | mcg |
| USP) | | |
| Copper (as Copper Sulfate) | approximately 0.2 | mg |
| L-Isoleucine (USP) | approximately 25 | mg |
| L-Leucine (USP) | approximately 50 | mg |
| L-Valine (USP) | approximately 25 | mg |
| Choline (as Choline Bitartrate, FCC) | approximately 25 | mg |
| Maltodextrin (as Maltrin M-100) | Q.S. | |

The beverage is prepared by heating a quantity of water to approximately 150° F. A sufficient amount of Raftilose and Fructose are added and mixed thoroughly until dissolved. A sufficient amount of Sucralose and Acesulfame-K are added and mixed thoroughly until dissolved. A quantity of premixture of the vitamins and minerals, and a sufficient amount of L-Arginine is added and mixed thoroughly until dissolved. Once the ingredients are thoroughly combined, flavoring and debittering agents are added. Next, a sufficient quantity of Citric Acid is added to adjust the pH to approximately 4.0.

Following blending of the beverage solution, the admixture is sterilized and homogenized. The solution is heated to approximately 200° F. for about 10 seconds at 1000 psi. While still hot, the solution is placed in bottles, sealed and quick cooled, by plunging a sealed bottle in an ice bath.

Each 8-ounce serving comprises the following approximate constituents:

| | | |
|---|---|---|
| Oligofructose-Raftilose P95-Pwd RHF | approximately 2.95 | g |
| Krystar Crystalline Fructose-Sweetener | approximately 5.79 | g |
| STC | | |
| Sweetener-Splenda MNP | approximately 0.064 | g |
| Acesulfame-K (Potassium)-Sweetener | approximately 0.027 | g |
| RCC | | |
| Premixture | approximately 225 | mg |
| Natural Flavor (OSF 1.01) | approximately 2.72 | g |
| Citric Acid-Anhydrous (Acidulent) HRC | approximately 6.5 | g |
| Water | approximately 581.7 | g |
| L-Arginine | approximately 3 | g |

The beverage is blended to provide the following approximate components:

| | |
|---|---|
| Sodium | 2% DV |
| Potassium | 2% |
| Copper | 10% |
| Chromium | 10% |
| Chorine | 25 mg |
| Boron | 1 mg |
| L-Arginine | 3000 mg |
| L-Leucine | 50 mg |
| L-Isoleucine | 25 mg |
| L-Valine | 25 mg |

Each 8-ounce serving contains the following approximate nutrients per serving:

| | |
|---|---|
| Calories | 53.27 |
| Protein | 0.38 g |
| Carbohydrates | 17.35 g |
| Dietary Fiber | 2.79 g |
| Fat - total | 0.00 g |
| Saturated Fat | 0 g |
| % calories from fat | 0% |
| % calories from carbs | 98% |
| Vitamin A RE | 0.12 RE |
| Vitamin C | 0.06 mg |

L-Arginine is known to be a human growth hormone precursor that stimulates the pituitary gland, which controls body growth. In a preferred embodiment, the beverage is consumed on a daily basis, preferably at night before sleeping. While the body is at rest, the HGH works to stimulate muscle growth naturally.

The invention has been described with references to a specific preferred embodiment. While specific values, relationships, materials and steps have been set forth for purposes of describing concepts of the invention, it will be appreciated by persons skilled in the art that numerous variations and/or modifications may be made to the invention as shown in the specific embodiments without departing from the spirit or scope of the basic concepts and operating principles of the invention as broadly described. It should be recognized that, in the light of the above teachings, those skilled in the art can modify those specifics without departing from the invention taught herein. Having now fully set forth the preferred embodiments and certain modifications of the concept underlying the present invention, various other embodiments as well as certain variations and modifications of the embodiments herein shown and described will obviously occur to those skilled in the art upon becoming familiar with said underlying concept. It is intended to include all such modifications, alternatives and other embodiments insofar as they come within the scope of the appended claims or equivalents thereof. It should be understood, therefore that the invention may be practiced otherwise than as specifically set forth herein. Consequently, the present embodiments are to be considered in all respects as illustrative and not restrictive.

1. A liquid beverage product for consumption by humans for restoring human growth hormone, said beverage product comprising:
(A) pantothenic acid;
(B) boron;
(C) chromium;
(D) copper;
(E) isoleucine;
(F) leucine;
(G) valine;
(H) choline;
(I) arginine; and
(J) water in a quantity at least sufficient for providing a solution wherein components (A) to (I) are substantially dissolved and which solution is ready for consumption by drinking.

2. The beverage product of claim 1, in which the isoleucine is in the form of L-Isoleucine, the leucine is in the form of L-Leucine, the valine is in the form of L-Valine, and the arginine is in the form of L-Arginine.

3. The beverage product of claim 1, wherein the ingredients consist of:
(A) about 2 mg to about 8 mg of pantothenic acid;
(B) about 0.5 mg to about 2 mg of boron:
(C) about 10 mcg to about 15 mcg of chromium;
(D) about 0.1 mg to about 0.4 mg of copper:
(E) about 15 mg to about 45 mg of L-Isoleucine;
(F) about 30 mg to about 75 mg of L-Leucine;
(G) about 15 mg to about 45 mg of L-Valine;
(H) about 15 mg to about 45 mg of choline; and
(I) about 3000 mg of L-Arginine.

4. The beverage product of claim 1, wherein the ingredients consist of:
(A) approximately 5 mg of pantothenic acid;
(B) approximately 1 mg of boron;
(C) approximately 12 mcg of chromium;
(D) approximately 0.2 mg of copper;
(E) approximately 25 mg of L-Isoleucine;
(F) approximately 50 mg of L-Leucine;
(G) approximately 25 mg of L-Valine;
(H) approximately 25 mg of choline; and
(I) approximately 3000 mg of L-Arginine.

5. A method of preparing a liquid beverage product for consumption by humans for restoring human growth hormone, said method comprising the steps of:
(A) heating a quantity of water to approximately 150° F.;
(B) adding a sufficient quantity of a natural sweetener and dissolving such sweetener in the water;
(C) adding a sufficient quantity of a premixture and dissolving such premixture in the water;
(D) adding a sufficient quantity of L-Arginine;
(E) after thoroughly dissolving all ingredients in the water, adding flavoring and debittering agents and a sufficient quantity of citric acid to adjust the pH to approximately 4.0.

6. The method of claim 5, in which such natural sweeteners are selected from the group consisting of:
(A) Raftilose;
(B) Fructose;
(C) Sucralose; and
(D) Acesulfame-K.

7. The method of claim 5, in which such premixture comprises:
(A) about 2 mg to about 8 mg of pantothenic acid;
(B) about 0.5 mg to about 2 mg of boron;
(C) about 10 mcg to about 15 mcg of chromium;
(D) about 0.1 mg to about 0.4 mg of copper;
(E) about 15 mg to about 45 mg of L-Isoleucine;
(F) about 30 mg to about 75 mg of L-Leucine;
(G) about 15 mg to about 45 mg of L-Valine;
(H) about 15 mg to about 45 mg of choline.

7. The method of claim 5, further comprising the steps of:
(I) sterilizing and homogenizing such solution; and
(J) dividing such solution into smaller quantities for bottling.

8. A method of supplementing the diet of a human to stimulate muscle growth comprising administering leucine, isoleucine, valine and arginine in combination with pantothenic acid, sodium, potassium, copper, chromium, chorine, and boron on a daily basis.

9. The method of claim 8 wherein the isoleucine is in the form of L-Isoleucine, the leucine is in the form of L-Leucine, the valine is in the form of L-Valine, and the arginine is in the form of L-Arginine.

10. The method of claim 8 wherein the arginine is administered in a dosage of approximately 3000 mg.

11. The method of claim 8 wherein the leucine is administered in a dosage of about 30 mg to about 75 mg.

12. The method of claim 8 wherein the isoleucine is administered in a dosage of about 15 mg to about 45 mg.

13. The method of claim 8 wherein the valine is administered in a dosage of about 15 mg to about 45 mg.

14. The method of claim 8 wherein the leucine is administered in a dosage of approximately 50 mg.

15. The method of claim 8 wherein the isoleucine is administered in a dosage approximately 25 mg.

16. The method of claim 8 wherein the valine is administered in a dosage of approximately 25 mg.
